(19) United States
(12) Patent Application Publication
(10) Pub. No.: US 2026/0199439 A1
(43) Pub. Date: Jul. 16, 2026

(54) THERAPEUTIC SUPPLEMENT

(71) Applicant: Manna Health LLC, San Diego, CA (US)

(72) Inventors: Rodney D. STRAMEL, San Diego, CA (US); Jeff HILL, San Diego, CA (US)

(73) Assignee: Manna Health LLC, San Diego, CA (US)

(21) Appl. No.: 19/026,138

(22) Filed: Jan. 16, 2025

(51) Int. Cl. A61K 38/39; A61K 9/127; A61K 47/10; A61K 47/12; A61K 47/24

(52) U.S. Cl. CPC A61K 38/39; A61K 9/127; A61K 47/10; A61K 47/12; A61K 47/24

(57) ABSTRACT

A nano-liposomal collagen supplement. The system also includes sunflower lecithin, oil, glycerin and water. The collagen supplement is processed such that the resulting solution is transparent and having a particle size less than 100 nm utilizing a high pressure homogenizer having a 16-reactor emulsifying cell.

The present application discloses compositions and methods to prepare supplements of non-water soluble chemicals. For example, the application discloses supplements including collagen into nano-liposomal supplements to provide improved bio-availability.

Nanoliposome, or submicron bilayer lipid vesicle, is employed to encapsulate and deliver various bioactive agents or compounds. Some low water-soluble supplements (e.g., collagen) are difficult to be incorporated into nanoliposomes. There is a need for a solution to permit low water-soluble chemicals or compounds to be incorporated into nano-lipsomes in order to take advantage of the improved bioavailability of nano-lipsomes supplements. Particularly, there is a demand for a stable nano-liposomal product for low water-soluble chemicals as supplements. The present application discloses such a solution and product.

The present application discloses a nano-liposomal supplement and method of making thereof, specifically nano-liposomal collagen and method of making a supplement employing these compounds.

As disclosed herein, collagen is made into a nano-liposomal supplement using solvent (glycerin), oil (MCT oil), and sunflower lecithin in water. The resulting product is a nano liposomal supplement that is transparent unlike other prior art collagen supplements. The supplement is transparent due to the nano sized particles created by the process disclosed herein. The end product is a liquid transparent supplement that can be consumed.

For the purpose of promoting an understanding of the principles of the disclosed compositions and methods, reference will now be made to embodiments illustrated herein and specific language will be used to describe the same. It will nevertheless be understood that no limitation of the scope of the invention is thereby intended. Any alterations and further modification in the described compositions, methods, products, and any further application of the principles of the invention as described herein, are contemplated as would normally occur to one skilled in the art to which the invention relates.

Nano liposomal technology enhances supplement delivery by encapsulating active ingredients within sub-100 nanometer lipid vesicles. These liposomes, with diameters less than 100 nanometers, readily penetrate cell membranes due to their nano-scale size. This enhanced cellular uptake significantly increases the bioavailability and efficacy of the encapsulated supplements compared to conventional delivery methods.

Water acts as the primary solvent with glycerin acting as a co-solvent, helping to dissolve both water-soluble and oil-soluble ingredients. Glycerin helps the oil and water emulsify thus creating a more homogeneous and stable mixture.

MCT oil forms the core of the liposomes, providing a hydrophobic environment to encapsulate the collagen particles.

Sunflower Lecithin acts as the primary emulsifier, helping to form stable liposomes by reducing surface tension between the water and oil.

The combination of the ingredients listed above is utilized in a unique method would result in a novel nano-liposomal collagen supplement disclosed herein.

### BRIEF DESCRIPTION OF THE DRAWINGS

FIG. 1 is a simplified diagram of an exemplary high pressure homogenizer.

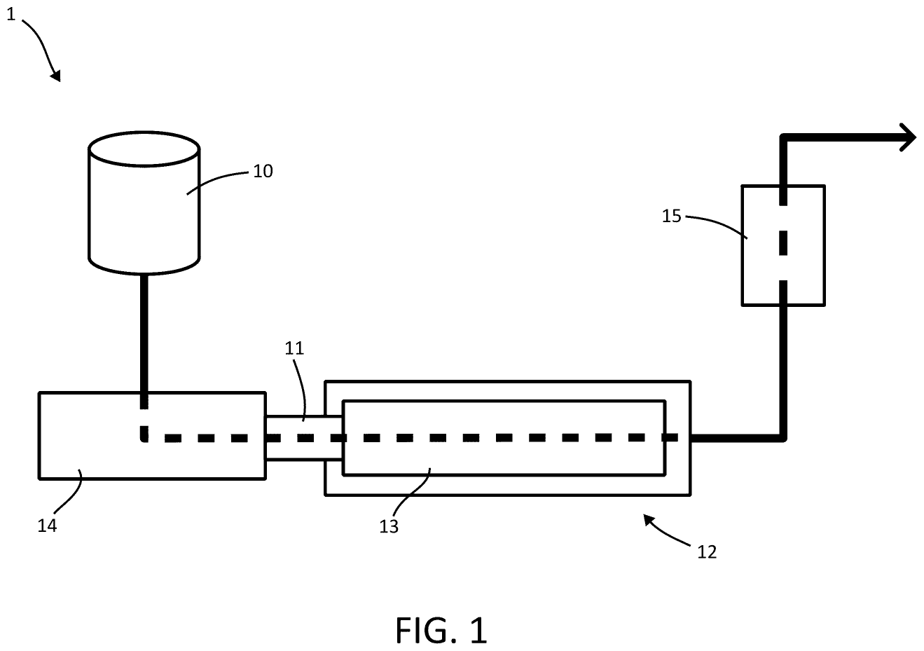

FIG. 2 is a simplified diagram of a reactor chamber of an emulsifying cell of the homogenizer in FIG. 1.

FIG. 3 is a close up of the reactors in series of the emulsifying cell as shown in FIG. 2.

### EXAMPLE FORMULATIONS

A first exemplary formulation of the nano-liposomal collagen supplement includes:

FORMULATION 1

| COMPONENT | % Weight |
|---|---|
| SUNFLOWER LECITHIN | 5.0 |
| BOVINE COLLAGEN | 45.0 |
| GLYCERINE | 3.00 |
| NATURAL SWEETENER | 0.75 |
| OIL | 1.00 |
| POTASSIUM SORBATE | 0.13 |
| SORBIC ACID | 0.07 |
| FLAVORING | 4.30 |
| OFF-NOTE FLAVOR MASKING | 0.30 |
| USP WATER | 40.5 |

The first formulation are mixed together to create a mixture. The mixture is then micro fluidized by a high pressure homogenizer (e.g. DeBee Mini 45) at a set pressure (e.g. 45000 psi).

Regarding Formulation 1, the utilized sunflower lecithin can be Alcolec H 20 (24%). The utilized collagen may be bovine collagen (e.g. New Zealand grass fed bovine). The utilized glycerine may be Glycerine 99.7% USP. The utilized natural sweetener may be made from Stevia Rebaudiana. The utilized oil may be MCT Oil (C8). The utilized off-note flavor masking can be any flavor modifiers that can provide flavor receptor blocking properties for unwanted flavors in functional ingredients such as excessive bitterness, metallic flavors, saltiness, sourness, etc. The off-note flavor masking can be ClearMask™ 201 NMP. The flavoring may be any type of flavor desired to be imparted to the supplement. For example, the flavoring can be natural chocolate milk flavoring.

A second exemplary formulation of the nano-liposomal collagen supplement includes:

FORMULATION 2

| COMPONENT | % Weight |
|---|---|
| SUNFLOWER LECITHIN | 5.00 |
| COLLAGEN | 10.0 |
| GLYCERIN | 3.00 |
| NATURAL SWEETENER | 0.60 |
| OIL | 1.00 |
| POTASSIUM SORBATE | 0.10 |
| SORBIC ACID | 0.10 |
| OFF-NOTE FLAVOR MASKING | 3.50 |
| USP WATER | 76.7 |

The second formulation are mixed together to create a mixture. The mixture is then micro fluidized by a high pressure homogenizer (e.g. DeBee Mini 45) at a set pressure (e.g. 45000 psi).

Regarding Formulation 2, the utilized sunflower lecithin can be Alcolec H 20 (24%). The utilized collagen may be vegan collagen powder or collagen made from fermented Pichia pastoris (e.g. Collawise™ vegan collagen powder). The utilized glycerine may be Glycerine 99.7% USP. The utilized natural sweetener may be made from Stevia Rebaudiana. The utilized oil may be MCT Oil (C8). The utilized off-note flavor masking can be any flavor modifiers that can provide flavor receptor blocking properties for unwanted flavors in functional ingredients such as excessive bitterness, metallic flavors, saltiness, sourness, etc. The off-note flavor masking can be ClearMask™ 201 NMP. The flavoring may be any type of flavor desired to be imparted to the supplement. For example, the flavoring can be natural chocolate milk flavoring. A third exemplary formulation of the nano-liposomal collagen supplement includes:

FORMULATION 3

| COMPONENT | % Weight |
|---|---|
| SUNFLOWER LECITHIN | 5.0 |
| MARINE COLLAGEN | 45.0 |
| GLYCERIN | 3.00 |
| NATURAL SWEETENER | 0.75 |
| OIL | 1.00 |
| POTASSIUM SORBATE | 0.13 |
| SORBIC ACID | 0.07 |
| FLAVORING | 4.30 |
| OFF-NOTE FLAVOR MASKING | 0.30 |
| USP WATER | 40.5 |

The third formulation are mixed together to create a mixture. The mixture is then micro fluidized by a high pressure homogenizer (e.g. DeBee Mini 45) at a set pressure (e.g. 45000 psi).

Regarding Formulation 3, the utilized sunflower lecithin can be Alcolec H 20 (24%). The utilized collagen may be marine/fish collagen (e.g. Nippi Peptide Wild Caught Marine Collagen). The utilized glycerine may be Glycerine 99.7% USP. The utilized natural sweetener may be made from Stevia Rebaudiana. The utilized oil may be MCT Oil (C8). The utilized off-note flavor masking can be any flavor modifiers that can provide flavor receptor blocking properties for unwanted flavors in functional ingredients such as excessive bitterness, metallic flavors, saltiness, sourness, etc. The off-note flavor masking can be ClearMask™ 201 NMP. The flavoring may be any type of flavor desired to be imparted to the supplement. For example, the flavoring can be natural chocolate milk flavoring.

A fourth exemplary formulation of the nano-liposomal collagen supplement includes:

FORMULATION 4

| COMPONENT | % Weight |
|---|---|
| SUNFLOWER LECITHIN | 5.0 |
| BOVINE COLLAGEN | 15.0 |
| POULTRY COLLAGEN | 15.0 |
| MARINE COLLAGEN | 15.0 |
| GLYCERIN | 3.00 |
| NATURAL SWEETENER | 0.75 |
| OIL | 1.00 |
| POTASSIUM SORBATE | 0.13 |
| SORBIC ACID | 0.07 |
| FLAVORING | 4.30 |
| OFF-NOTE FLAVOR MASKING | 0.30 |
| USP WATER | 40.5 |

The fourth formulation are mixed together to create a mixture. The mixture is then micro fluidized by a high pressure homogenizer (e.g. DeBee Mini 45) at a set pressure (e.g. 45000 psi).

Regarding Formulation 4, the utilized sunflower lecithin can be Alcolec H 20 (24%). The utilized collagen may be three types of collagen (e.g. bovine, poultry, and marine). The bovine collagen maybe New Zealand grass fed bovine. The utilized poultry collagen may be type 2 chicken collagen. The marine collagen may be wild caught marine/fish collagen (e.g. Nippi Peptide Wild Caught Marine Collagen). The utilized glycerine may be Glycerine 99.7% USP. The utilized natural sweetener may be made from Stevia Rebaudiana. The utilized oil may be MCT Oil (C8). The utilized off-note flavor masking can be any flavor modifiers that can provide flavor receptor blocking properties for unwanted flavors in functional ingredients such as excessive bitterness, metallic flavors, saltiness, sourness, etc. The off-note flavor masking can be ClearMask™ 201 NMP. The flavoring may be any type of flavor desired to be imparted to the supplement. For example, the flavoring can be natural chocolate milk flavoring.

It should also be noted that the natural sweetener, flavoring, and off-note flavor masking can be grouped as flavor modifiers.

FORMULATION CHARACTERISTICS -
SINGLE PASS (16 REACTOR)

| | Mv | Mn | Ma | D50 | D95 |
|---|---|---|---|---|---|
| FORMULATION 1 | 49 nm | 40 nm | 46 nm | 47 nm | 75 nm |
| FORMULATION 2 | 36 nm | 24 nm | 29 nm | 29 nm | 75 nm |
| FORMULATION 3 | 16 nm | 11 nm | 13 nm | 13 nm | 31 nm |
| FORMULATION 4 | 46 nm | 31 nm | 39 nm | 41 nm | 83 nm |

Mv=Volume mean diameter (De Brouckere Mean Diameter).
Mn=Number mean diameter.
Ma=Surface area mean dimeter (Sauter Mean Diameter).
D50=50^{th} percentile diameter.
D95=95^{th} percentile diameter.

Volume mean diameter (Mv, De Brouckere Mean Diameter, or D[4,3]) is the is the mean of a particle size distribution weighted by the volume.

Number mean diameter (Mn, or D[1,0]) is the average diameter of the particle per the number of particles.

Surface area mean dimeter (Ma, Sauter Mean Diameter, or D[3,2]) is the mean diameter with the same volume to surface area ratio as the sample.

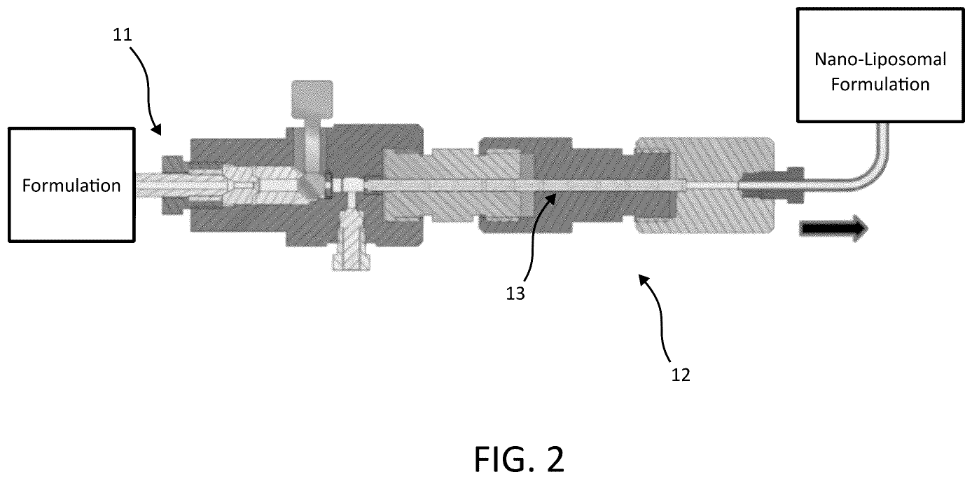

The formulations are prepared using a high-pressure homogenizer 1 utilizing a novel 16-reactor emulsifying cell 12. The emulsifying cell incorporates an alternating pattern of 1 mm and 0.5 mm inner diameter straight-bore ceramic reactors, enabling the production of very small particles with only a single pass. The emulsifying cell may be configured in a parallel flow configuration, such that the flow travels only in one direction rather than in two directions in a reverse flow set up. As seen in FIG. 2 the mixture is passed through left to right from the reservoir 10 through the processing nozzle 11 via a pump 14 as a high velocity jet stream and is directed to the emulsifying cell 12 having a series of reactors 13. Inside the series of reactors 13, the product will undergo forces (e.g. shearing, cavitation, turbulence, etc.) in order to create the nano-liposomal particles. This configuration results in smaller particles than current known solutions.

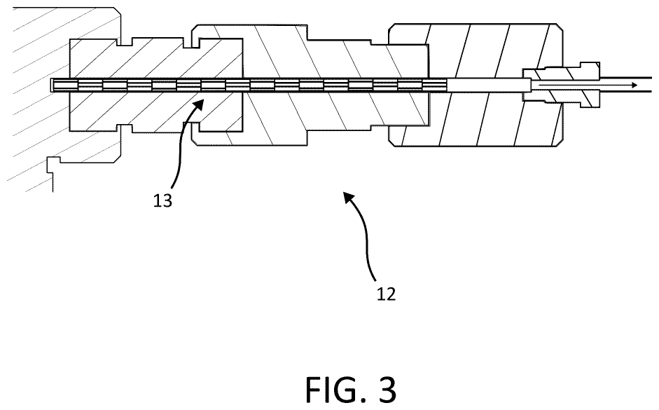

FIG. 3 depicts the emulsifying cell, which utilizes sixteen co-axial reactors 13 in an alternating 1 mm/0.5 mm diameter arrangement. The formulation then passes through cooling chamber 15 before exiting the homogenizer. This precise number of reactors has been found to achieve minimal particle size in a series flow system without requiring a large emulsifying cell. The end product is a liquid transparent supplement that can be consumed.

A nano-lipsomal collagen supplement is disclosed herein. The supplement preferably includes the following components specified below:
about 5% by weight sunflower lecithin;
about 45% by weight collagen;
about 3% by weight glycerin;
about 0.75% by weight sweetener;
about 1% by weight oil;
about 0.13% by weight potassium sorbate;
about 0.07% by weight sorbic acid; and
about 40.5% or more by weight water.

In another embodiment, a nano-lipsomal collagen supplement is disclosed herein. The supplement preferably includes the following components specified below:
about 5% by weight sunflower lecithin;
about 10% by weight collagen;
about 3% by weight glycerin;
about 0.60% by weight sweetener;
about 1% by weight oil;
about 0.1% by weight potassium sorbate;
about 0.1% by weight sorbic acid; and
about 76.7% or more by weight water.

A nano-lipsomal collagen supplement is disclosed herein. The supplement preferably includes the following components specified below:
about 5% by weight sunflower lecithin;
about 15% by weight bovine collagen;
about 15% by weight poultry collagen;
about 15% by weight marine collagen;
about 3% by weight glycerin;
about 1% by weight oil;
about 0.13% by weight potassium sorbate;
about 0.07% by weight sorbic acid; and
about 40.5% or more by weight water.

It is to be understood that while a certain form of the invention is described, it is not intended to be limited to the specific form or arrangement herein described. It will be apparent to those skilled in the art that various changes may be made without departing from the scope of the invention and the invention is not to be considered limited to what is shown and described in the specification.

One skilled in the art will readily appreciate that the present invention is well adapted to carry out the objectives and obtain the ends and advantages mentioned, as well as those inherent therein. The compositions, formulations, methods, and techniques described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention are intended to be within the scope of the invention.

What is claimed is:

1. A transparent nano-liposomal collagen supplement made from a 16-reactor emulsifying cell comprising the following ingredients:
5% by weight sunflower lecithin;
45% by weight collagen;
3% by weight glycerin;
1% by weight oil;
0.13% by weight potassium sorbate;
0.07% by weight sorbic acid;
40.5% or more by weight water; and
wherein the ingredients are passed through the emulsifying cell to thereby create nano-liposomal particles.

2. The supplement of claim 1, further comprising 5.35% by weight flavor modifier.

3. The supplement of claim 1, wherein the flavor modifier includes:
0.75% by weight sweetener;
4.30% by weight flavoring; and
3% by weight off-note flavor masking.

4. The supplement of claim 1, wherein the collagen is a combination of at least one of bovine collagen, poultry collagen, and marine collagen.

5. The supplement of claim 1, wherein the mean diameter of the nano-liposomal collagen is less than 100 nm.

6. The supplement of claim 1 wherein the mean diameter of the mean diameter of the nano-liposomal collagen is 22 nm to 30 nm.

7. The supplement of claim 1, wherein the surface mean diameter of the nano-liposomal collagen is between 26 nm to 34 nm.

8. The supplement of claim 1, wherein the volume mean diameter of the nano-liposomal collagen is between 29 nm to 41 nm.

9. A transparent nano-liposomal collagen supplement made from a 16-reactor emulsifying cell comprising:
5% by weight sunflower lecithin;
10% by weight collagen;
3% by weight glycerin;
1% by weight oil;
0.13% by weight potassium sorbate;
0.07% by weight sorbic acid;
76.7% or more by weight water; and,
wherein the ingredients are passed through the emulsifying cell to thereby create nano-liposomal particles.

10. The supplement of claim 9, further comprising 5.35% by weight flavor modifier.

11. The supplement of claim 9, wherein the flavor modifier includes:
0.75% by weight sweetener;
4.3% by weight flavoring; and
3% by weight off-note flavor masking.

12. The supplement of claim 9, wherein the collagen is vegan collagen.

13. The supplement of claim 9, wherein the mean diameter of the nano-liposomal collagen is less than 100 nm.

14. The supplement of claim 9, wherein the mean diameter of the mean diameter of the nano-liposomal collagen is 24 nm.

15. The supplement of claim 9, wherein the surface mean diameter of the nano-liposomal collagen is 29 nm.

16. The supplement of claim 9, wherein the volume mean diameter of the nano-liposomal collagen is 36 nm.

17. A high pressure homogenizer comprising:
a reservoir comprising a mixture;
a pump configured to receive said mixture;
a high pressure nozzle downstream of the pump;
an emulsifying cell receiving said mixture via the high pressure nozzle, wherein the emulsifying cell includes a plurality of reactors arranged in sequence in a series flow configuration.

18. The homogenizer of claim 17, wherein the plurality of reactors include at least 16 reactors.

19. The homogenizer of claim 17, wherein the reactors alternate in diameter so that one of the reactors has a first diameter and that one reactor is positioned in series between two reactors, wherein each of the two reactors has a second diameter that is greater than the first diameter.

20. The homogenizer of claim 17, wherein the reactors alternate in diameter so that one of the reactors has a first diameter and that one reactor is positioned in series between two reactors, wherein each of the two reactors has a second diameter that is less than the first diameter.
